(19) United States
(12) Patent Application Publication
(10) Pub. No.: US 2024/0036026 A1
(43) Pub. Date: Feb. 1, 2024

(54) DYSBIOTIC MEASUREMENT

(71) Applicant: GLYCIOME, LLC, Valleyford, WA (US)

(72) Inventors: Joanna E. ELLINGTON, Valleyford, WA (US); G. D. CLIFTON, Valleyford, WA (US); Rayne ELLINGTON-LAWRENCE, Valleyford, WA (US)

(73) Assignee: GLYCIOME, LLC, Valleyford, WA (US)

(21) Appl. No.: 18/479,756

(22) Filed: Oct. 2, 2023

Related U.S. Application Data
(63) Continuation of application No. PCT/US2022/023200, filed on Apr. 2, 2022.
(60) Provisional application No. 63/170,296, filed on Apr. 2, 2021.

(51) Int. Cl. G01N 33/487

(52) U.S. Cl. CPC G01N 33/48707

(57) ABSTRACT

The present disclosure provides systems and methods for the identification of dysbiotic biological samples.

### CROSS REFERENCE TO RELATED APPLICATIONS

The present application claims priority to U.S. App. No. 63/170,296, filed Apr. 2, 2021, which is hereby incorporated by reference in its entirety.

### BACKGROUND

The genital microbiome of a human is a unique combination of microbial species comprising at least one hundred species of bacteria and a variety of fungal, viral, and protozoal species. There is considerable variation in make-up of the genital microbiome between individuals, with many factors such as hygiene regimes, diet, environment, age, ethnicity, disease, sexual activity, sexual orientation, and life history affecting the presence of specific microbial species and their metabolic activities. Environmental conditions within the genitalia compared to other locations of the human body are distinct. As a result, products indicated for urogenital, anogenital, vaginal and/or penile use, to modulate the genital microbiome, may require distinct prebiotic and/or probiotic constituents.

Additionally, the genital microbiome is unique in that it is shared between members of a sexual dyad and between mammalian mother and newborn as a primary mode of the newborn's establishment of its own microbiome. Diseases and/or dysbiosis within the dyad can occur if a healthy genital microbiome is not supported. Microbiome congruency and transfer of microbial species between sexual partners, and between a mother and her child is well documented. Sexual partners can transmit pathogenic bacteria back and forth that harm beneficial bacteria, increase inflammation, and increase risk for disease, including infertility, reproductive dysfunction, poor pregnancy outcomes, autoimmune disease, sexually transmitted diseases (e.g., HIV, HSV, HPV), cancers (e.g., prostate, cervical), and systemic diseases such as cognitive impairment.

In particular, bacterial vaginosis (BV) is a common bacterial imbalance of the vaginal microbiome. As the genital microbiome is often shared between sexual partners, the penis often has an effect on the bacterial balance of the vagina. For example, correlations between BV incidence and penile microbiota have been shown which suggest the exchange of BV− associated bacteria between sexual partners through intercourse. Furthermore, male genital dysbiosis (e.g., penile growth of the same organisms that cause BV) may be etiologically connected with various sexually transmitted diseases such as HIV, HSV, or HPV, urethritis, abnormal sperm quality, and penile cancers.

The pathogenesis of vaginal and penile chronic dysbiosis, rely on the production of bioactive amines. A few species of dysbiotic bacteria (e.g., the bacteria present in a microbiome under dysbiosis) produce the most significant amounts of biogenic amines such as agmatine, putrescine, cadaverine, and tyramine, which increase vaginal pH, harm beneficial Lactobacillus spp and make the vaginal microbiome more inviting to other dysbiotic species. These species may also produce bacterial endotoxins such as lipopolysaccharides which have been linked to local and systemic diseases, disorders, and conditions such as various cancers and dementia. Vaginal dysbiosis is often linked to concurrent male partner penile dysbiosis. This shift in metabolic states from a glycogen/carbohydrate to amino acid-rich environment results in a shift in vaginal pH away from the beneficial acidic state (e.g. pH of from 4 to 5 such as 4.5) to a more alkaline state. Such shift is initiated and perpetuated by specific bacteria including species from the genera: pathogenic Prevotella, Eggerthellia, Gardnerella, Atopobium, Megasphaera, Mobiluncus, Mageeibacillus, Gemella, Veillonella, Snethia, Mycoplasma, and some Clostridium species. It is important to note that some of these bacteria are not most abundant or solely present in a dysbiotic microbiome, confirming that the metabolic activity and the ecological role that a microbe plays in an environment is more important than its abundance. These dysbiotic bacteria produce metabolites that result in increased genital pH, offensive odors, biofilm production that interferes with host immune function, increased epithelial inflammation (which may result in symptoms such as burning, itching, and/or pain), increased mucin degradation (which may result in symptoms such as pain, roughness of the skin, and/or irritation), increased oxidative stress, cytokine and inflammasome production, and decreased growth capacity of healthy microbiome organisms. These conditions leave the individual susceptible to the development of further dysbiosis and disease states, including increased risk of poor reproductive outcomes (e.g., failed fertilization, failed embryo implantation, poor fetal growth, delivery complications), pain during sex, sexually transmitted diseases (e.g., high risk Human Papilloma virus (HPV) infection and persistence, genital Herpes Simplex Virus (HSV) infection, and Human Immunodeficiency Virus (HIV) infection), auto-immune conditions, cancer, and systemic disease (e.g., cognitive impairment). These conditions also have a social cost of embarrassment and social withdrawal for women with odor and discharge and for men with odor and visual roughness or inflammation of penile skin. These social aspects of genital dysbiosis are rarely discussed and under-treated.

Existing cleaning products and genital therapies (e.g., body washes, wipes, yeast treatments, most lubricants) have pH, salt levels and ingredients that harm genital tissues, gametes, and kill healthy bacteria. For example, a popular, commercially available diaper wipe has a pH of 3 while the World Health Organization (WHO) has stated that this pH level is inconsistent with human genital tissues. Furthermore, measurement driven application regimens are often lacking and current modalities are insufficient. For example, over the counter (OTC) pH tests are usually nitrazine paper-based, with color scales that are difficult or impossible (16% of the time) to match to manufacturer's scale, depend on lighting and eyesight, only have moderate agreement between users and change if moved from the physiologic environment (vagina). Due to the great complexity of substituents in the microbiome, it is difficult to ascertain those components typically present in dysbiotic microbiomes as compared to healthy ones.

There remains a need for effective and accurate measurements of microbiomes that can allow for reliable identification of dysbiosis for subsequent measurement driven application regimens.

### SUMMARY

The present disclosure provides sensors, systems, and methods affording measurement of the microbiome. In particular, by leveraging certain biological sample measurement systems as described herein, the detection of various parameters of the microbiome can be elucidated giving insight into possible detrimental metabolic states, that for example, induce the growth of pathogenic metabolomic species and/or decrease the growth of beneficial metabolomic species. These methods may involve measurements of the metabolomic profile and/or pH of these bodily regions in an attempt to inform the treatment regimen (e.g., when to administer, which type of composition to deliver, etc.). In another aspect, the disclosure herein provides an integrated penile pH tracking system for the measurement of penile pH.

The systems for the measurement of one or more parameters of the metabolomic profile of a biological sample (e.g., derived from saliva, blood, urine, feces, genital fluids, tears, nasal swabs, sweat, psoriasis lesions). These systems may be used in concert with the methods described herein. In some embodiments, the system may comprise:
a substrate;
a sensor medium immobilized on said substrate comprising a plurality of carbon nanostructures; wherein the plurality of carbon nanostructures have one or more conductive materials deposited thereon;
at least two conductive terminals in electrical connection with the sensor medium and spaced from each other;
at least one measurement system to measure one or more electrical properties of the sensor medium when the sensor medium comprises the biological sample deposited thereon; and
a correlation system calibrated to correlate the measured electrical property with the one or more parameters of the metabolomic profile of the biological sample.

For example, the correlation system may correlate the measured electrical property to the pH and/or the concentration of one or more of cadaverine, putrescine, and tyramine. In particular embodiments, the correlation system may correlate the measured electrical property to the pH and/or the summed concentration of cadaverine, putrescine, and tyramine. By applying specific electric potentials across the sensor medium having the nanostructure deposited thereon and measuring parameters associated therewith, the correlation system may be able to identify the metabolomic state based on the measured parameters. Typically, the correlation system has been trained through machine learning algorithms in order to convert the measured parameters into an identification of specific metabolomic profiles such as the identification of concentrations of biogenic amines as described herein.

These systems may be used in devices for measurements of the metabolomic profile and/or pH of biological samples. In some embodiments, the identification of dysbiosis involves measurements of the pH and the metabolomic profile of the biological sample. In some embodiments, the device for the measurement of one or more parameters of the metabolomic profile of a biological sample (e.g., derived from saliva, blood, urine, feces, genital fluids, tears, nasal swabs, sweat, psoriasis lesions) may comprise:
a) a handle portion dimensioned to be held in a user's hand comprising a power source (e.g., one or more batteries such as a lithium ion battery, one or more solar cells); and
b) a sensor portion comprising one or more sensors; wherein the sensors comprise:
   a substrate;
   a sensor medium immobilized on said substrate comprising a plurality of carbon nanostructures; wherein the plurality of carbon nanostructures has one or more conductive materials deposited thereon;
   at least two conductive terminals in electrical connection with the sensor medium and spaced from each other;
wherein said sensor portion is removably attached to the handle portion; and when the sensor portion is attached to the handle portion, the one or more systems are in electrical communication with the power source;
and said device comprises at least one measurement system to measure one or more electrical properties of the sensor medium when the sensor medium comprises the biological sample deposited thereon.

Methods for the measurement of one or more parameters of the metabolomic profile of a biological sample (e.g., derived from saliva, blood, urine, feces, genital fluids, tears, nasal swabs, sweat, psoriasis lesions) are also provided which may comprise:
depositing or collecting the biological sample on a sensor medium comprising a plurality of carbon nanostructures; wherein the plurality of carbon nanostructures has one or more conductive materials deposited thereon;
measuring an electrical property of the sensor medium after depositing the biological sample; and
correlating the measured electrical property to the one or more parameters of the metabolomic profile.

For example, the correlation system may correlate the measured electrical property to the pH and/or the concentration of one or more of cadaverine, putrescine, and tyramine. In particular embodiments, the correlation system may correlate the measured electrical property to the pH and/or the summed concentration of cadaverine, putrescine, and tyramine. These systems, devices, and methods of measurement may also be used with administration regimens. For example, some embodiments for the method of optimizing the beneficial microbiome growth in the genital region of a subject in need thereof may comprise:
measurement of one or more parameters of the metabolomic profile of a biological sample with the presently disclosed measurement systems and/or devices and/or methods;
application of a pharmaceutical composition having an acidic pH to the genital region of the subject or to the genital region of a sexual partner of the subject in need thereof based on the one or more parameters of the metabolomic profile.

In some embodiments, the pharmaceutical compositions (e.g., rinses, lavages, douches, serums, topical, topical isotonic, gels, lubricants) to be administered comprise:
(a) a metallic co-factor (e.g., manganese chloride);
(b) a prebiotic oligosaccharide (e.g., lactulose); and
(c) borneol or a prodrug thereof (e.g., bornyl acetate);
wherein the composition may be buffered with a buffer system comprising gluconolactone. In particular, the compositions may provide an increased buffering capacity in a pH range to support beneficial microbiome optimization for a person in need thereof (e.g., in the vaginal microbiome). The compositions may, for example, have increased buffering capacity in a pH range of from 3.5-7 or from 4-7 or from 5-7 or from 5-6 or from 3-6. In some embodiments, the compositions of the present disclosure may be delivered using vaginal or topical films wherein the composition is capable of diffusing from the film into its surrounding environment. Suitable film formers include chitosan, hydroxypropyl methylcellulose and blends of these polymers (e.g., with 40% PEG 400 as plasticizer), a polymeric matrix/chitosan with carrageenan (κ-, λ-, and τ-), pectin and gellan gum, hydroxyl propylcellulose and sodium alginate as polymers and propylene glycol and polyethylene glycol-400 as plasticizers, polyvinyl alcohol, poloxamer 407 and 188, hypromellose, sodium carboxymethylcellulose, hydroxylpropylmethylcellulose, hydroxyethylcellulose and polyvinyl pyrrolidone K-90, hydroxypropyl methylcellulose and Eudragit polymers (e.g., Eudragit RL100) and propylene glycol as plasticizer, hydroxypropyl methylcellulose, polyvinyl alcohol, polyethylene oxide, glycerol, poly(2-oxazoline)/polyoxazoline polymers and combinations thereof.

### DETAILED DESCRIPTION

Prior to setting forth this disclosure in more detail, it may be helpful to an understanding thereof to provide definitions of certain terms to be used herein. Additional definitions are set forth throughout this disclosure.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. Any concentration ranges recited herein are to be understood to include concentrations of any integer within the range and fractions thereof, such as one tenth, one hundredth, and one thousandth of an integer, unless otherwise indicated. Unless otherwise indicated, it will be understood that any percentage refers to the weight percentage with respect to the indicated component. Typically, the percent of a component in the composition indicates the weight percentage with respect to the weight of the composition.

The term “consisting essentially of” is not equivalent to “comprising” and refers to the specified materials or steps, or to those that do not materially affect the basic and novel characteristics of the claimed subject matter. It should be understood that the terms “a” and “an” as used herein refer to “one or more” of the enumerated components.

The genital microbiota or genital microbiome may include the collective microorganisms that normally colonize the genital region. The genital microbiota may be non-pathogenic. The genital microbiota may refer to that of the genital skin microbiota, the vaginal microbiota (e.g., vaginal mucosal microbiota) of a female subject, the cervical microbiota of a female subject, penile microbiota (e.g., penile skin microbiota such as the foreskin microbiota or the urethral meatus microbiota) of a male subject, microbiota of the genital tissue of an intersex individual, microbiota of a non-binary gendered individual, or any combination thereof. Recently, species overlap between rectal and urinary microbiome species in an individual have been observed. For example, the bacteria of the genital region have been found to represent a continuum between organs of excretion and reproduction as discussed Y. Govender, et al., Front Cell Infect Microbiol 9 (2019): 133, hereby incorporated by reference in its entirety and particularly in relation to the urinary microbiomes disclosed therein. These bacteria of the genital region are referred to herein as the anogenital and/or urogenital microbiomes.

Genital probiotic bacteria may refer to live bacteria, which when administered in adequate amounts to the vagina or penis confer a health benefit (e.g., such as those described herein) to the host subject.

The vaginal microbiota or vaginal flora refers to the collective microorganisms that normally colonize the vulva, clitoris, vestibule, and vagina and are typically non-pathogenic. In general, beneficial vaginal microbiota is primarily comprised of different strains of Lactobacillus (or related acid-producing bacterial types), which produce lactic acid to keep the vaginal ecosystem at a tightly controlled acidic environment (e.g., pH of 3.5-5.5 or 3.5-5) during much of a woman's monthly cycle in reproductive aged women. An exemplary vaginal microbiome includes a dominance of healthy Lactobacillus species including: Lactobacillus jensenii, Lactobacillus gasseri, and Lactobacillus crispatus. Other beneficial species may include Lactobacillus acidophilus, Lactobacillus plantarum, Lactobacillus fermentum, Lactobacillus brevis, Lactobacillus casei, Lactobacillus delbrueckii, Lactobacillus vaginalis, Lactobacillus salivarius, Lactobacillus reuteri, and Lactobacillus rhamnosus. Additional bacterial species often present in lower numbers in the vaginal region of healthy women include: Lactobacillus iners, and species from the genuses Prevotella, Megasphaera, Colstridium, Baccilus, Gardnerella, Sneathia, and Mycoplasma. Additionally, acid producing bacteria that may be part of the normal vaginal microbiota in some women including Lactobacillus iners, and species of Prevotella, Atopobium, Leptotrichia, Leuconostoc, Megasphaera, Pediococcus, Streptococcus, and Weissella. The species found in normal vaginal microbiota can vary depending on age and ethnicity. In some ethnicities and life stages, the acid-producing, healthy vaginal microbiome may not be dominated by Lactobacillus spp. Evidence has shown that Lactobacillus spp. dominance is associated with optimal reproductive and genital health outcomes in women of all ages. Various microbiota (and their connection with both vaginal and non-vaginal health) are disclosed in J. Si, et al., Cell Host & Microbe 21 (2017): 97-105, hereby incorporated by reference in its entirety, and particularly in relation to vaginal microbiota including Lactobacillus and Prevotella species.

Reproductive tract microbiota may include the collective microorganisms that normally colonize the female upper reproductive tract, such as the cervix, uterus, Fallopian tubes, and ovaries, and are non-pathogenic. In certain embodiments, the female reproductive tract microbiota can also be comprised of species from the genus Pseudomonas, Acinetobacter, Vagococcus, Sphingobium, Erysipelothrix, Facklamia or Prevotella. In certain embodiments, application of the present compositions may have differential effect on species in the reproductive tract microbiota. This differential effect may be measured, for example, with the systems and methods of the present application before and/or during and/or after an application regimen. For example, application of the present compounds may result in increases or maintained levels on beneficial bacteria such as healthy Lactobacillus species such as L. crispatus, L. jensenii, and L. gasseri. In some embodiments, administration of the compositions of the present disclosure may inhibit the growth of pathobionts such as those from the Gardnerella genus (e.g., G. vaginalis) or the Prevotella genus (e.g., P. bivia). In some embodiments, administration may result in increases or maintained levels on beneficial bacteria such as healthy Lactobacillus species such as L. crispatus. L. jensenii, and L. gasseri and inhibition of the growth of pathobionts such as those from the Gardnerella genus (e.g., G. vaginalis) or the Prevotella genus (e.g., P. bivia) for example, as measured between 12 and 48 hours following administration. In some embodiments, administration of the present compositions may result in increases or maintained levels on beneficial bacteria such as healthy Lactobacillus species such as L. crispatus, L. jensenii, and L. gasseri and reduction of pathobionts in the microbiome including one or more pathobionts such as those from the Gardnerella genus (e.g., G. vaginalis) or the Prevotella genus (e.g., P. bivia) for example, as measured between 12 and 48 hours following administration.

The vaginal microbiota is often affected by the penile microbiota and correlations between BV and penile microbiota between members of a sexual dyad have been shown in C. Liu, et al., mBio 6 (2015): e00589-15, hereby incorporated by reference in its entirety and particularly in relation to the exchange of BV associated bacteria through intercourse and connections between Nugent score and penile community state types. Penile microbiota or penile flora may refer to the collective microorganisms that normally colonize the penis, foreskin, and distal urethra which are typically non-pathogenic. The penis includes the penile shaft and distal glans, which includes the glans, glans coronal, meatus urethralis, fossa navicularis, frenulum, coronal sulcus, and foreskin. In various implementations, the penile microbiota, and in particular, in an optimized penile microbiome, comprises bacterial species from the genus Lactobacillus, Streptococcus, Staphylococcus, Corynebacteria, and combinations thereof. In certain embodiments, the penile microbiota comprises Lactobacillus crispatus, Lactobacillus jensenii, Lactobacillus iners, Lactobacillus gasseri, Streptococcus, non-pathogenic Prevotella, Corynebacteria, Staphylococcus, Anaerococcus, Peptoniphilus, Finegoldia, Porphyromonas, Propionibacterium, Delftia, Bfidobacterium, Clostridium, non-pathogenic Pseudomonas, or any combination thereof. In certain embodiments, the penile microbiota of a male subject reflects the vaginal microbiota and/or reproductive tract microbiota of a female subject, wherein the male subject and female subject are members of a sexual dyad. The species found in normal penile microbiota can differ between circumcised and uncircumcised subjects. For example, penile microbiota may also include sperm microbiota such as those disclosed in D Baud, et al., Frontiers in Microbiol 10 (2019): 234, hereby incorporated by reference in its entirety and particularly in relation to beneficial seminal microbiota including Lactobacillus species. In some embodiments, the compositions are able to increase the number of beneficial species in the penile microbiome (and by extension the vaginal microbiome following intercourse) such as Lactobacillus, Streptococcus, Staphylococcus, Corynebacteria, and optimize or balance (e.g., decrease and/or increase) the number of dysbiosis associated anaerobes such as those from the species Prevotella, Finegoldia, Diallister, Snethia, Megasphaeae, Mobiluncus, Mycoplasma, Peptococcus, Peptostreptococcus, Porphyromonas, Slackia, Tannerella, Treponema, Ureaplasma, Veillonella, Actinobacteria, Anaerococcus, Actinomyces, Aggregatibacter, Atopobium, Bacteroides, Bifidobacteriium, Clostridiales, Eggerthella, Eubacterium, Fusobacterium, Gardnerella, Leptotrichia, and combinations thereof. In some embodiments, optimization of the microbiome involves decreasing the number of pathogenic communities of bacteria such as those species from the genera Gardnerella, Finegoldia, Dialister, Prevotella, Anaerococcus, Atopobium, Megasphaera, and combinations thereof.

The anogenital region of a subject includes regions of the anus and the genitalia. In certain embodiments, the female anogenital region comprises the cervix, vagina, vulva, clitoris, urethral meatus, urethral meatus, vulval vestibule, perineum, and/or anus. In certain embodiments, the male anogenital region comprises the penis, base of the penis, foreskin, urethral meatus, scrotum, perineum, and anus. The term urogenital region may refer to the region of the distal urinary tract and the genitalia. In certain embodiments, the female urogenital region comprises the cervix, vagina, vulva, clitoris, introitus, urethral meatus, urethral fold, vulval vestibule, and/or perineum. In some subjects, the anogenital and/or urogenital regions of a subject may be indistinct, intersex, or transitioning from male to female or female to male due to iatrogenic (e.g., surgery or hormone therapy) or natural/genetic causes.

Genital tissues are often living cells found in the anogenital and/or urogenital regions. Genital tissues include, but are not limited to epithelial surface cells (e.g., skin), mucosal cells, immune cells, nerve cells, blood cells, connective tissue cells, and neoplastic cells of the vulva, clitoris, vagina, vestibule, vulval vestibule, urethral meatus, penis, foreskin, distal urethra, and scrotum. Since sperm cells exit the penis and are often deposited on genital tissue (e.g., vagina), genital tissues also include semen and sperm cells.

Genital fluids are often secretions from the body that naturally occur in and around genital tissues. Genital fluids include, but are not limited to, cervical and vaginal secretions (together often referred to as cervico-vaginal fluids (CVF)), semen, smegma, seminal fluid, urethral secretions, epithelial and mucosal coatings, menses flow, post-partum lochia, amniotic fluid, and other fluids naturally occurring in and around the vagina, vulva, clitoris, penis, foreskin, and scrotum. Gametes may be found in genital fluids. For example, male gametes may be found in male genital fluids such as semen, smegma, seminal fluid, and/or urethral secretions or fluid.

The reproductive cycle or menstrual cycle is a cycle of hormone changes comprising both the production of an oocyte (ovarian cycle) and preparation of the uterus for pregnancy (uterine cycle). A cycle of the reproductive cycle may include the time of peak fertility in the cycle immediately before and after ovulation as well as the period when conception is not possible due to the lack of a viable egg.

“Effective amount” or “therapeutically effective amount” refers to that amount of a composition of this disclosure which, when administered to a subject, such as a human, is sufficient to affect a desired biological effect or treatment including optimizing of the penile microbiome. In some embodiments, the effective amount may have minimal effect on gametes following application (e.g., with minimal changes in motility, concentration, vitality, morphology of gametes, oxidation-reduction potential, sperm DNA fragmentation, sperm mitochondrial membrane potential, survival, and changes at the sub-cellular levels such as changes to proteins related to specific functions of the gametes). In some embodiments, the therapeutically effective amount alters one or more features of gametes in the genital fluids of the user by less than 20%.

As used herein, the term “subject” refers to any organism to which a composition in accordance with the disclosure may be administered, e.g., for experimental, diagnostic, prophylactic, and/or therapeutic purposes. Typical subjects include any animal (e.g., mammals such as mice, rats, rabbits, non-human primates, and humans, etc.). A subject in need thereof is typically a subject for whom it is desirable to treat a disease, disorder, or condition as described herein (and in particular, treatment of a disease, disorder or condition relating to dysbiosis of the urogenital and/or anogenital regions). For example, a subject in need thereof may seek or be in need of treatment, require treatment, be receiving treatment, may be receiving treatment in the future, or a human or animal that is under care by a trained professional for a particular disease, disorder, or condition.

As used herein, the phrase “pharmaceutically acceptable” generally safe for ingestion or contact with biologic tissues at the levels employed. Pharmaceutically acceptable is used interchangeably with physiologically compatible.

The compounds described herein may be present as a pharmaceutically acceptable salt. Typically, salts are composed of a related number of cations and anions (at least one of which is formed from the compounds described herein) coupled together (e.g., the pairs may be bonded ionically) such that the salt is electrically neutral. Pharmaceutically acceptable salts may retain or have similar activity to the parent compound (e.g., an ED₅₀ within 10%) and have a toxicity profile within a range that affords utility in pharmaceutical compositions. For example, pharmaceutically acceptable salts may be suitable for use in contact with the tissues of humans and animals without undue toxicity, irritation, allergic response and are commensurate with a reasonable benefit/risk ratio. Pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. Salts may be prepared from pharmaceutically acceptable non-toxic acids and bases including inorganic and organic acids and bases. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, dichloroacetate, digluconate, dodecylsulfate, ethanesulfonate, formate, fumarate, glucoheptonate, glutamate, glycerophosphate, hemisulfate, heptonate, hexanoate, hippurate, hydrobromide, hydrochloride, hydroiodide, 2-hydroxy-ethanesulfonate, isethionate, lactobionate, lactate, laurate, lauryl sulfate, malate, maleate, malonate, mandelate, methanesulfonate, mucate, 2-naphthalenesulfonate, nicotinate, nitrate, oleate, oxalate, palmitate, pamoate, pantothenate, pectinate, persulfate, 3-phenylpropionate, phosphate, picrate, pivalate, propionate, stearate, succinate, sulfate, tartrate, thiocyanate, toluenesulfonate, undecanoate, and valerate salts. Representative basic salts include alkali or alkaline earth metal salts include sodium, lithium, potassium, calcium, and magnesium, aluminum salts, as well as nontoxic ammonium, quatemary ammonium, and amine cations, including, but not limited to ammonium, tetramethylammonium, tetraethylammonium, methylamine, dimethylamine, trimethylamine, triethylamine, caffeine, and ethylamine.

Pharmaceutically acceptable acid addition salts of the disclosure can be formed by the reaction of a compound of the disclosure with an equimolar or excess amount of acid. Alternatively, hemi-salts can be formed by the reaction of a compound of the disclosure with the desired acid in a 2:1 ratio, compound to acid. The reactants are generally combined in a mutual solvent such as diethyl ether, tetrahydrofuran, methanol, ethanol, iso-propanol, benzene, or the like. The salts normally precipitate out of solution within, e.g., one hour to ten days and can be isolated by filtration or other conventional methods.

Prodrugs are typically compounds that may be converted under physiological conditions or by solvolysis to a biologically active compound of the invention. Prodrug may refer to a metabolic precursor of a compound of the invention that is pharmaceutically acceptable. A prodrug may be inactive when administered to a subject in need thereof, but is converted in vivo to an active compound of the invention. Prodrugs are typically rapidly transformed in vivo to yield the indicated compound, for example, by hydrolysis in blood. The prodrug compound often offers advantages of solubility, tissue compatibility or delayed release in a mammalian organism as described in Bundgard, H., Design of Prodrugs (1985): 7-9, 21-24 (Elsevier, Amsterdam), Higuchi, T., et al., ACS Symposium Series, Vol. 14, and Bioreversible Carriers in Drug Design, Ed. Edward B. Roche, American Pharmaceutical Association and Pergamon Press, 1987, each of which are hereby incorporated by reference in their entirety.

The methods provided in the present disclosure may be used on an animal subject (e.g., mammalian, bovine, canine, feline, equine, porcine, ovine, avian, rodent, lagomorph, caprine, non-human primate), preferably a human subject. In certain embodiments, the subject is a male, a female, an intersex subject, a non-binary gendered subject, or a subject of any other gender designation. In certain embodiments, the subject is an infant, a child, or an adult. In certain embodiments, the subject is an adult male of reproductive age (e.g., ranging 18 years to 50 years) that is trying to conceive or adult female of reproductive age (e.g., ranging from 18 years to 50 years) that is trying to conceive. In certain embodiments, the subject is a female in menopause or male in andropause. In certain embodiments, the subject is an infant (aged from 0 to 12 months old), a child at least 1 year old; an adult ranging from 18 years to 50 years of age, 18 to 55 years of age, or 18 to 60 years of age; or a senior adult of at least 50 years, at least 55 years, at least 60 years, or at least 65 years of age. In certain embodiments, a senior is at least 60 years old. In certain embodiments, a senior is at 70 years old. In certain embodiments, a senior is at least 80 years old.

The biological samples (e.g., saliva, blood, urine, feces, genital fluids, tears, nasal swabs, sweat, psoriasis lesions) may be obtained from the animal subject and deposited onto the sensing medium for measurements of the metabolic profile associated therewith. In some embodiments, the biological sample may be manipulated (e.g., diluted with, for example, normal saline) prior to deposition in order to associate the biological sample with the intended correlation system. It will be understood that “derived from” in relation to any biological samples includes unaltered biological samples taken directly from a subject and biological samples that have undergone manipulation (e.g., diluted) prior to deposition on the sensing medium. The biological sample may be deposited on the substrate by direct contact with a specific portion of the subject (e.g., inside the vagina, on penis) or with a material produced by the subject (e.g., genital fluids, sweat, saliva, blood, urine, feces, tears, mucus such as nasal mucus). The present disclosure is partially based on the discovery of specific biogenic amines which correlate with dysbiosis of biological samples (e.g., derived from saliva, blood, urine, feces, genital fluids, tears, nasal swabs, sweat, psoriasis lesions). Additionally, correlations between the electrical signals from these systems and the required biogenic amines may be determined by machine learning algorithms which allow for facile diagnosis of dysbiosis. Such machine learning algorithms include linear discriminate analysis optionally in combination with support vector machine (SVM), k-nearest neighbors, random forest and combinations thereof. The systems may comprise:
a substrate;
a sensor medium immobilized on said substrate comprising a microfluidic chip and/or a plurality of carbon nanostructures; wherein the plurality of carbon nanostructures have one or more conductive materials deposited thereon;
at least two conductive terminals in electrical connection with the sensor medium and spaced from each other;
at least one measurement system to measure one or more electrical properties of the sensor medium when the sensor medium comprises the biological sample deposited thereon; and
a correlation system calibrated to correlate the measured electrical property with the one or more parameters of the metabolomic profile of the biological sample.

Typically, these systems operate using electric field as field effect transistors. Accordingly, the carbon nanostructure deposited on the sensing medium may exhibit a doping level or electronic shift upon interaction with a biological sample absorbed thereon. Such shifts may manifest in various changes to the electronic interaction of the carbon structure resulting in measurable electronic differences. For example, the electrical properties of the sensor may change in transconductance, threshold voltage shift, relative change in conductance at a specified voltage (e.g., from −1V to 1V), change in overall conductance when normalized to the threshold voltage, and the relative change in the minimum conductance, or combinations thereof as compared to the sensor medium not having the biological sample deposited thereon or in in situ contact. These various electrical properties may be measured converted into an assessment of the metabolomic profile following input into the correlation system which has been trained to correlate the measured parameters with aspects of the metabolic state. In some embodiments, deposition of the biological sample occurs through in situ direct or indirect contact with the sensing medium. Furthermore, these various changes in the electronic nature of the sensor medium may be correlated (e.g., through linear discriminate analysis of the measured electronic changes on the sensor medium to determine and train which variables or measurements are correlated with the metabolomic profile of the biological sample (and in particular, biological samples correlated with distinct bodily microbiomes such as cervical fluids)) with electrical properties and measurements performed on a biological sample dispersed on the sensing medium in order to analyze and detect parameters of the metabolic profile in a biological sample.

The plurality of carbon nanostructure may comprise one or more carbon nanotubes (e.g., single walled carbon nanotube) and/or graphene sheets (e.g., holed graphene). In some embodiments, the graphene comprises zigzag edge states. In some embodiments, the carbon nanostructure comprises armchair edge states. In some embodiments, the carbon nanostructure is oxidized.

Further tuning of the electronic properties may be achieved through deposition of various materials onto the plurality of carbon nanostructures. For example, the plurality of carbon nanostructures may have one or more conductive materials deposited thereon. In some embodiments, the conductive materials may be selected from one or more conductive polymers (e.g., polyaminoanthracene, polyaniline, polypyrrole) or one or more conductive metal (e.g., silver, gold, palladium, platinum) or combinations thereof. In some embodiments, the conductive material may comprise at least two conductive metals in the form of nanoparticles deposited on the plurality of carbon nanostructures. In certain implementations, the conductive material may comprise two or more forms of metal nanoparticles; wherein the forms of metal nanoparticles differ by surface functionalization. Such surface functionalization may comprise self-assembled monolayers of compounds having different head groups. The head groups may be independently selected from alkyl (e.g., C₁-C₃ alkyl such as methyl), —OH, —COOH, —SH, and —NRR; wherein R is independently selected at each occurrence from hydrogen and alkyl. In certain implementations, one form of metal nanoparticle comprises a self-assembled monolayer of dodecanethiol. In some embodiments, the form of metal nanoparticle comprises 11-mercaptounedecanoic acid.

As discussed above, different permutations and functionalization of the carbon nanomaterial may have effect on the electroscopic properties of the system. These can be measured and correlated with biological samples. For example, the measured electrical properties may comprise a change in transconductance, threshold voltage shift, relative change in conductance at a specified voltage (e.g., from −1V to 1V), change in overall conductance when normalized to the threshold voltage, and the relative change in the minimum conductance, or combinations thereof as compared to the sensor medium not having the biological sample deposited thereon. In some embodiments, the correlation between the one or more biological samples and their metabolomic profile has been determined by linear discriminant analysis (LDA). In some embodiments, the identification of dysbiosis in a biological sample may have occurred by linear discriminate analysis (e.g., in combination with the support vector machine algorithm) on healthy and/or dysbiotic samples which may identify correlation parameters for the system. In some embodiments, the identification of dysbiosis in a biological sample may have occurred by linear discriminate analysis (e.g., in combination with the support vector machine (SVM) algorithm). In some embodiments, correlation between healthy and dysbiotic biological samples and/or training of the sensors may have occurred by linear discriminate analysis in combination with one or more of SVM, k-Nearest Neighbors (k-NN), random forest, or combinations thereof.

These sensor mediums of the present disclosure may also be configured to measure the pH of the biological sample based on the electrical properties of the sensor medium having the biological substance deposited thereon or in contact in situ.

The systems may involve the transmission of data to an external device. For example, in some embodiments, the correlation system may comprise the transmission of the electrical property to an external device (e.g., laptop, tablet, computer, server, smart phone). In various implementations, the correlation system may comprise the transmission of data to an external device (e.g., laptop, tablet, computer, server, smart phone) configured to correlate the transmitted data into the one or more parameters of the metabolomic profile.

In certain implementations, the one or more parameters of the metabolomic profile may be correlated to the number of one or more species of pathogenic bacteria present in the biological sample, the number of one or more species of beneficial bacteria present in the biological sample, similarity to a metabolomic profile of a biological sample from a healthy individual (e.g., eubiotic individuals, individuals without hrHPV), similarity to a metabolomic profile of a biological sample from an individual suffering a particular disease, disorder, or condition (e.g., dysbiosis, urethritis, bacterial vaginosis, hrHPV), or combinations thereof. The metabolomic profile may comprise, or relate to, one or more biogenic amines such as cadaverine, putrescine, spermine, spermidine, agmatine, tyrosine, tyramine, cadaverine, agmatine, N-acetylputrescine, camitine, deoxycamitine, pipecolic acid, pipecolate, lactate, tyrosine, sphingosine, adenine, guanine, xanthine, uric acid, caffeine, glutamate, phenylalanine, glutathione, glycylproline, or combinations thereof. In certain implementations, the metabolomic profile may comprise, or relate to, one or more amino acids and molecules similar thereto such as arginine, betaine, choline, lysine, methionine, omithine, and S-adenosyl methionine and/or short chain fatty acids such as formate, acetate, propionate, butyrate, isobutyrate, valerate, and isovalerate and/or vitamins or vitamin-like compounds such as vitamin B2, vitamin B12, vitamin C, vitamin A, vitamin D, vitamin K, vitamin B3, and vitamin B5 and/or antioxidants such as glutathione. The sensor array may measure metabolomic profiles of those compounds which are produced chemically and/or microbiologically including Vitamins A and C, those compounds which are produced in one or more microbial-enzymatic step including Vitamins B3, B5, D, and K, and those compounds produced by bacterial fermentation (e.g., neurotransmitter compounds) including γ-aminobutyric acid (GABA), 5-HT (serotonin), acetylcholine, dopamine and histamine. Several of these components such as Vitamins D, K, B3, and B5 may be measured individually, in groups thereof, or collectively by the systems of the present disclosure. The sensors may identify differences the concentration of any one biogenic amines, amino acids and molecules similar thereto, short chain fatty acids, antioxidants, vitamin-like compounds, compounds produced chemically in the microbiome or microbiologically, compounds produced in one more microbial-enzymatic steps, compounds produced by bacterial fermentation, or combinations thereof over time, or in comparison to a baseline or control. In some embodiments, the sensor may identify the concentration or changes concentration (e.g., with respect to a reference, a sample, a control, a baseline) of one or more of cadaverine, putrescine, spermine, spermidine, agmatine, tyrosine, tyramine, cadaverine, agmatine, N-acetylputrescine, camitine, deoxycamitine, pipecolic acid, pipecolate, lactate, tyrosine, sphingosine, adenine, guanine, xanthine, uric acid, caffeine, glutamate, phenylalanine, glutathione, glycylproline, arginine, betaine, choline, lysine, methionine, omithine, and S-adenosyl methionine, formate, acetate, propionate, butyrate, isobutyrate, valerate, and isovalerate, vitamin B2, vitamin B12, vitamin C, vitamin A, vitamin D, vitamin K, vitamin B3, and vitamin B5, or combinations thereof. In some embodiments, the sensor may identify the summed concentration of cadaverine, putrescine, and tyramine in the biological sample. In some embodiments, the sensor has a pH electrode as well. In some embodiments, whether the microbiome needs further optimization may be determined based on sensing of specific metabolites associated with disease, disorder, or condition, such as the biomarkers of bacterial vaginosis described, for example, in T Nelson, Frontiers in Physiology 6 (2015): Article 253, which is hereby incorporated by reference in its entirety and particularly in relation to the biomarkers and concentrations thereof such as those identified in FIGS. 1, 3, 5, 6, and Table 4. In some embodiments, the metabolomic profile of a biological sample may be assessed by the sensors and systems of the present disclosure. In some embodiments, the biological sample may be obtained from the subject and placed on the sensing medium for analysis.

In some embodiments, any metabolite such as those described herein may be measured in the form produced in situ, or as conjugate acids thereof, conjugate bases thereof, oxidized forms thereof, or reduced forms thereof.

The measurement of the metabolomic profile of the biological sample may occur with one or more microfluidics devices as the sensing medium or in concert with the nanocarbon FET based devices described herein. The microfluidic sensing portion may comprise a microfluidic chip comprising: at least one channel, and a detector configured to detect signals of metabolites within the channel. These microfluidics devices may use colorimetric or electrostatic based measurement, such as those described in A Koh, et al., Sci Transl Med 8 (2016): 366dra165 which is hereby incorporated by reference in its entirety and particularly in relation to microfluidics designs and protocols. The microfluidic sensors may identify metabolites present in the biological sample such as biogenic amines, amino acids and molecules similar thereto, short chain fatty acids, vitamin-like compounds, compounds produced chemically in the microbiome or microbiologically, compounds produced in one more microbial-enzymatic steps, compounds produced by bacterial fermentation, or combinations thereof over time, or in comparison to a baseline or control. In some embodiments, the microfluidics device may measure the concentration of metallic ions such as metal co-factor that may comprise zinc, selenium, manganese, molybdenum, cobalt, iron, copper, including salts thereof, or any combination thereof. Microfluidics devices may be based on chemiluminescent reactions with, for example, luminol based reactants, such as those described in C Provin, et al., IEEE Jour of Oceanic Engineering 38 (2013): 178-185, which is hereby incorporated by reference in its entirety and particularly in relation to chemiluminescent microfluidic sensing.

Devices for the measurement of one or more parameters of the metabolomic profile of a biological sample (e.g., derived from saliva, blood, urine, feces, genital fluids, tears, nasal swabs, sweat, psoriasis lesions) are also provided. These devices may comprise:
a) a handle portion dimensioned to be held in a user's hand comprising a power source (e.g., one or more batteries such as a lithium ion battery, one or more solar cells, USB connection, RFID); and
b) a sensor portion comprising one or more sensors; wherein the sensors comprise:
   a substrate;
   a sensor medium immobilized on said substrate comprising a microfluidics-based sensor and/or a plurality of carbon nanostructures; wherein the plurality of carbon nanostructures has one or more conductive materials deposited thereon;
   at least two conductive terminals in electrical connection with the sensor medium and spaced from each other;
wherein said sensor portion is removably attached to the handle portion; and when the sensor portion is attached to the handle portion, the one or more systems are in electrical communication with the power source; and
said device comprises at least one measurement system to measure one or more electrical properties of the sensor medium when the sensor medium comprises the biological sample deposited thereon or in in situ contact therewith. In some embodiments, the power source is an electrical conduit between another device which provides the power required for measurement such as USB connections or RFID. In some embodiments, the device may be configured to transmit the electrical property to an external device (e.g., laptop, tablet, computer, server, smart phone). In various implementations, the device may further comprise a correlation system calibrated to correlate the measured electrical property with the one or more parameters of the metabolomic profile of the biological sample. In some embodiments, the device may comprise one or more of the systems described herein. For example, the device may comprise from 1-1000 (e.g., 1-500, 3-500, 3-100) sets of substrates, sensor medium, and electrical contacts each independently selected in terms of surface functionalization, carbon nanostructure type, substrate material, contact with electrodes and the like. In some embodiments the device may comprise a microprocessor and computer storage medium (e.g., hard drive), wherein correlation parameters are stored in the storage medium and instructions for measurements provided to the power source connected to the electrodes and processed in the microprocessor in communication with the storage medium. In some embodiments, the device is connected to a correlation system external from the device, such that the measurement system transmits information to the correlation system to perform a reading on the biological sample and identify metabolomic profiles. The device may communicate the data (e.g., with Bluetooth, radio frequency identification, USB such as USB-A, USB-B, USB-C, micro USB, lightning cable) from the measurement system to an external device such as a laptop, tablet, computer, server, and/or smart phone comprising the instructions to correlate the data with metabolic profiles using, for example, a correlation system of the present disclosure.

The present disclosure also includes applicators which may be used for administration of a composition to the urogenital and/or anogenital region of a subject (e.g., the vagina, the penis) comprising a device for measurement of the metabolomic profile as described herein. The applicator may comprise a storage portion having an internal reservoir capable of storing one or more doses of the compositions of the present disclosure. In some embodiments, the internal reservoir may have a volume of from 0.01 μL to 60 mL or 0.01 μL to 5 mL or 0.01 μL to 1 mL or 1 mL to 60 mL or 1 mL to 5 mL or 0.01 μL to 10 μL or 0.01 μL to 100 μL or 0.1 μL to 1 mL or 0.1 μL to 5 mL or 5 mL to 60 mL or 10 mL to 50 mL or 15 mL to 30 mL or 20 mL to 25 mL. The internal reservoir may be in fluid communication with an application portion configured to release an amount of the composition. For example, a user may apply a force to the storage portion causing the composition to be expelled therethrough.

The applicator may further comprise a sensor capable of measuring one or more characteristics of the surrounding environment (e.g., the penis, the vagina) including the pH. For example, the applicator may comprise a litmus or nitrazine dye which, following insertion, is capable of visually displaying pH information of the environment of the urogenital and/or anogenital regions. In some embodiments, the applicator may comprise a nanosensor such as that disclosed in U.S. Pat. No. 10,436,745, hereby incorporated by reference in its entirety and particularly in relation to pH nanosensors. In some embodiments, the nanosensor is capable of measuring the pH of the surrounding environment (e.g., the anogenital and/or urogenital region). Following measurement, the nanosensor may be capable of communicating the pH measurement (e.g., with Bluetooth, radio frequency identification, USB such as USB-A, USB-B, USB-C, micro USB, lightning cable) with an external device such as a laptop, tablet, computer, server, and/or smart phone. The nanosensor may transmit the value of the measured pH or information relating to the pH. For example, the sensor may transmit a binary signal and/or a ternary signal depending on user settings. The external device may be configured to interpret, display, and track such measurements.

Another embodiment may provide an applicator comprising a nanosensor, such as a solid-state sensor based on oxidized (ox-) single-walled carbon nanotubes (ox-SWNTs) functionalized with a conductive polymer (e.g., poly(1-aminoanthracene) (PAA)). The nanosensor may, in some embodiments, have a Nernstian response over a wide pH range (e.g., pH 2-pH 12) and retain sensitivity for an extended period of time from first use or exposure to air such as over or up to 120 days (e.g., over or up to 60 days, over or up to 40 days). Another embodiment may provide for an applicator comprising a nanosensor attached to a passively-powered radio-frequency identification (RFID) tag, which may transmit information (e.g., pH data) to a mobile or portable device (e.g., tablet, smart phone) accessible through a software application or to a computer having the software application. This battery-less, reference electrode-free, wirelessly transmitting sensor platform may be used for biomedical applications, including but not limited to, intravaginal pH measurement or penile pH measurement, such as when attached to an applicator or probe. In some embodiments, the sensor platform includes a system for communicating with an external device such a blue-tooth communication system, a physical connection to an external device such as USB or lightning cable. In some embodiments, power is delivered to the sensing medium through the physical connection to the external device.

The applicators (or any portion thereof such as the container and/or the applicator element) may be formed from those materials known in the art. In some embodiments, portions of the applicator or the entire applicator may be made low waste packaging materials such as biodegradable plastics. Suitable biodegradable plastics may be bio-based plastics such as polyhydroxyalkanoates (PHAs), polylactic acid (PLA), starch blends, cellulose-based plastics, lignin-based polymer composites, and combinations thereof. The biodegradable plastics may also be petroleum based such as polyglycolic acid (PGA), polybutylene succinate (PBS), polycaprolactone (PCL), poly(vinyl alcohol) (PVA, PVOH), polybutylene adipate terephthalate (PBAT), and combinations thereof. In some embodiments, portions of the applicator (e.g., the applicator element) or the entire applicator may be composed of paper and/or cardboard. In some embodiments, the paper and/or cardboard applicators or portions thereof, may be burned or disposed of after vaginal mucosal contact, thereby decreasing risky medical waste.

### EXAMPLES

### Example 1: Sensor Measurements

Genital (vaginal and penile) pH is measured with oxidized single-walled carbon nanotubes functionalized with conductive polymer poly(1-aminoanthracene) (PAA) to detect surface pH level by changes in sensor conductance in response to protonation and deprotonation of carboxylic groups of the nanotube and amine groups of the polymer. The sensor is housed in a disposable sheath, integrated with a reusable handheld device, which connects with a purpose-built e-health app for routine genital health status updates, even in users with low healthcare fluency. The integrated e-health app may suggest over the counter (OTC) available management strategies, counseling with remote platform healthcare staff, or sharing of data and referral to user's healthcare providers which may be based on the information received from the sensor. Key novel factors of the sensor system may include: 1) ability to tailor the sensor for specific regional or ethnic groups (one size does not fit all), 2) inclusion of testing for men to improve community-wide reproductive health, and 3) real-time, low-cost assessment of genital health in diffuse, less accessible communities, for development of evidence-based solutions to intransient healthcare challenges.

The carbon nanomaterials to be used in the sensor may sense pH changes in contact with the genital surface as local charge density via functional groups on the PAA are protonated/deprotonated as described in P Gou, et al., Sci Rep 4 (2014): 4468, hereby incorporated by reference in its entirety. These changes alter conductance of the SWCNTs and are then measured via interdigitated electrodes on prefabricated silicon chips made via standard photolithography.

An alternative nanocarbon material, holey graphene (HG) as described in DL White, et al., Nano Lett 19 (2019): 2824-2831 and Y Xu et al., Nano Lett 15 (2015): 4605-4610, each of which is hereby incorporated by reference in their entirety and particularly in relation to graphene, holey graphene, and oxidized (ox-) forms thereof. Briefly, graphene, holey graphene, and oxidized forms thereof achieves similar changes as ox-SWCNT via edge localized moieties including hydroxyl and carboxylic groups and will also be evaluated for the system as described in H Vedala, Nano Lett 11 (2011): 2342-2347, hereby incorporated by reference in its entirety. These carbon nanomaterials are exceedingly sensitive (high surface to volume ratios and superior electrical conductance) to electrical measurements. Furthermore, carbon nanosensors are advantageous due to high chemical and thermal stability, reversibility of sensing response, high sensitivity, and ability to measure with biological sample contact (e.g., inside the vagina, on penis, of genital fluids) as described in D Kauffman et al., Angew Chem Int Ed Engl 47 (2008): 6550-6570, V Schroeder, et al., Chem Rev 119 (2019): 599-663, and M. Meyyappan Small 12 (2016): 2118-2129, each of which is hereby incorporated by reference in their entirety.

The carbon nanomaterial tubes may mitigate the issues of current user-directed pH monitoring, as data can be collected in the biologically relevant environment. In particular, the high chemical stability of nanomaterials is utilized, and may need only initial calibration at fabrication. These sensors may measure resistance changes in the pH sensor, convert the resistance to pH, and display this pH on the prototype screen, either for smartphone camera capture or Bluetooth integration with a purpose-built-health app. Real-time genital symptoms or events may be assessed (new partner, menses, new product) and inform the user of status and any need for medical follow up.

The sensor includes an easy-grip reusable handle with housing. The handle is removed prior to use and a disposable soft sheath containing the single-use nanosensor for pH measurement and metabolomic profiling is snapped on. The sensor may then be placed against a body portion such as the distal vaginal wall or penile surface (at the corona sulcus) for reading via integrated e-health app (e.g., Bluetooth or RFID signaling) which integrates measured outcomes with contextual user symptom/sexual wellness data. The sensor may also comprise a binary and/or ternary signal display system. A sensor of the present application may interface with a smart phone where a disposable sheath comprising the sensor unit, after measurement of biological sample from the user, may be removably attached to the handle portion. The handle sensor unit may then transmit the required information to an external device which provides recommendations to the user based on the measured data.

Sensor arrays composed of non-specific chemistry in combination with machine learning algorithms may be used to achieve selectivity through data analytics. This sensor array approach can be used to diagnose bacterial vaginosis.

As discussed in G Silva, et al., ACS Sens 2 (2017): 1128-1132, hereby incorporated by reference in its entirety, cell discrimination of carbon-based sensors has been demonstrated by utilizing a non-specific binding array sensor approach utilizing SWCNT FET devices decorated with gold nanoparticles decorated with different self-assembled monolayers (SAMs). Briefly, cells were immobilized on SWCNT devices and the FET response to the presence of cells was measured. Due to differences in cell membrane composition as well as excreted extracellular material, the response data of the cells studied to identify the unique characteristics of each cell type. These regions were defined by training the device with a large sample size of known identity and applying linear discriminant analysis (LDA) optionally including SVM, k-NN, or random forest algorithms to separate these regions-based sensor responses acquired from each type. By combining analysis of carbon-based FET device characteristics with supervised machine learning algorithms, successfully discrimination among five selected purine compounds was possible. These interactions of purine compounds with metal nanoparticle-decorated SWCNTs were further corroborated by density functional theory (DFT) calculations.

The metabolomic profiles consistent with high-risk genital dysbiosis in men and women in is performed using these techniques.

### Example 2: Establishing Correlations Between Sets of Biogenic Amines and Dysbiosis in Biological Samples

Metal nanoparticle-decorated SWCNT-FET devices, and linear discriminant analysis with support vector machine (SVM) algorithm was used to classify biogenic amine profiles in mock vaginal fluids based on health (i.e. BV+ or BV−). The SWCNT-FET sensors with varying build, tested differential responses from the “vaginal fluid” analytes. Defined solutions represent mean biogenic amine profiles from women diagnosed as bacterial vaginosis positive (BV+) or bacterial vaginosis negative (BV−). The dataset was stratified into a training and test sets with 20+ sensor measurements/sample to train and then test the model. Features were selected based on the nanoparticle-nature of the sensing material, correlations to the target amine profile, and between features, to train the SVM classifier. The classifier was tuned to balance the model bias and variance using training data. Then the SVM classifier was tested with test data, as summarized in a confusion matrix.

The biogenic amines cadaverine, putrescine, and tyramine (and particularly, their summed concentrations) were shown to be highly correlated with healthy and dysbiotic microbiomes. These biogenic amines were consistently measured in test samples to identify BV+ and BV− in biological samples.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet and Request are incorporated herein by reference, in their entirety. Aspects of the embodiments can be modified, if necessary to employ concepts of the various patents, applications, and publications to provide yet further embodiments.

These and other changes can be made to the embodiments in light of the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

1. A system for the measurement of one or more parameters of the metabolomic profile of a biological sample:
a substrate;
a sensor medium immobilized on said substrate comprising a microfluidic chip and/or a plurality of carbon nanostructures; wherein the plurality of carbon nanostructures have one or more conductive materials deposited thereon;
at least two conductive terminals in electrical connection with the sensor medium and spaced from each other;
at least one measurement system to measure one or more electrical properties of the sensor medium when the sensor medium comprises the biological sample deposited thereon; and
a correlation system calibrated to correlate the measured electrical property with the one or more parameters of the metabolomic profile,
wherein the one or more parameters of the metabolomic profile include the concentration of cadaverine, putrescine, and/or tyramine of the biological sample.

2. The system according to claim 1, wherein the plurality of carbon nanostructures comprises a carbon nanotube and/or graphene.

3. The system according to claim 1, wherein the carbon nanostructures comprises an oxidized nanostructure.

1. The system according to claim 1, wherein the conductive materials are selected from conductive polymers or a conductive metal or combinations thereof.

5. The system according to claim 1, wherein the conductive material comprises at least two conductive metals in the form of nanoparticles.

6. The system according to claim 1, wherein the conductive material comprises two or more forms of metal nanoparticles; wherein the forms of metal nanoparticles differ by surface functionalization.

7. The system according to claim 6, wherein the surface functionalization comprises self-assembled monolayers of compounds having different head groups.

8. The system according to claim 7, wherein the different head groups are independently selected from alkyl, —OH, —COOH, —SH, and —NRR; wherein R is independently selected at each occurrence from hydrogen and alkyl.

2. The system according to claim 5, wherein one form of metal nanoparticle comprises (or is functionalized with) a self-assembled monolayer of dodecanethiol and another form of metal nanoparticle comprises (or is functionalized with) 11-mercaptoundecanoic acid.

10. The system according to claim 1, wherein the one or more measured electrical properties comprise change in transconductance, threshold voltage shift, relative change in conductance at a specified voltage, change in overall conductance when normalized to the threshold voltage, and the relative change in the minimum conductance, or combinations thereof as compared to the sensor medium not having the biological sample deposited thereon.

11. The system according to claim 1, wherein the substrate is also configured to measure the pH of the biological sample based on the electrical properties of the sensor medium having the biological substance deposited thereon.

12. The system according to claim 1, wherein the correlation system comprises the transmission of the electrical property to an external device.

13. The system according to claim 1, wherein the correlation system comprises the transmission of data to an external device configured to correlate the transmitted data into the one or more parameters of the metabolomic profile.

14. (canceled)

15. The system according to claim 1, wherein the one or more parameters of the metabolomic profile is correlated with the number of one or more species of pathogenic bacteria present in the biological sample, the number of one or more species of beneficial bacteria present in the biological sample, similarity to a metabolomic profile of a biological sample from a healthy individual, similarity to a metabolomic profile of a biological sample from an individual suffering a particular disease, disorder, or condition, or combinations thereof.

16. A method for the measurement of dysbiosis of a biological sample comprising:
depositing the biological sample on a sensor medium comprising a plurality of carbon nanostructures; wherein the plurality of carbon nanostructures has one or more conductive materials deposited thereon;
measuring an electrical property of the sensor medium after depositing the biological sample; and
correlating the measured electrical property to the concentration of cadaverine, putrescine, and tyramine, or combinations thereof, wherein said correlation indicates the dysbiosis of the biological sample.

17. A method of optimizing the beneficial microbiome growth in the genital region of a subject in need thereof comprising:
measurement of one or more parameters of the metabolomic profile of a biological sample with the system according to claim 1;
application of a pharmaceutical composition having an acidic pH to the genital region of the subject or to the genital region of a sexual partner of the subject in need thereof based on the one or more parameters of the metabolomic profile.

18. A device for the measurement of one or more parameters of the metabolomic profile of a biological sample comprising:
a) a handle portion dimensioned to be held in a user's hand comprising a power source; and
b) a sensor portion comprising one or more sensors; wherein the sensors comprise:
a substrate;
a sensor medium immobilized on said substrate comprising a plurality of carbon nanostructures; wherein the plurality of carbon nanostructures has one or more conductive materials deposited thereon;
at least two conductive terminals in electrical connection with the sensor medium and spaced from each other;
wherein said sensor portion is removably attached to the handle portion; and when the sensor portion is attached to the handle portion, the one or more systems are in electrical communication with the power source; and
said device comprises at least one measurement system to measure one or more electrical properties of the sensor medium when the sensor medium comprises the biological sample deposited thereon.

19. The device according to claim 18, wherein said device is configured to transmit the electrical property to an external device.

20. The device according to claim 18, wherein said device further comprises a correlation system calibrated to correlate the measured electrical property with the one or more parameters of the metabolomic profile of the biological sample;
wherein said correlation system correlates the measured electrical property to the pH and/or the concentration of one or more of cadaverine, putrescine, and tyramine in the biological sample.

21. The device according to claim 4, wherein said correlation system correlates the measured electrical property to the pH and/or the summed concentration of cadaverine, putrescine, and tyramine in the biological sample.
